(19) United States
(12) Patent Application Publication
(10) Pub. No.: US 2010/0010221 A1
(43) Pub. Date: Jan. 14, 2010

(54) PROCESSES FOR PURIFYING VARENICLINE L-TARTRATE SALT AND PREPARING CRYSTALLINE FORMS OF VARENICLINE L-TARTRATE SALT

(76) Inventors: Revital Lifshitz-Liron, Herzlia (IL); Shalom Shabat, Yavne (IL); Sharon Tomer, Tel Aviv (IL); Yuriy Raizi, Natanya (IL); Eyal Gilboa, Qiryat Ono (IL); Pramod Kumar Pandey, Faridabad (IN)

Correspondence Address: KENYON & KENYON LLP, ONE BROADWAY, NEW YORK, NY, 10004, US

(21) Appl. No.: 12/473,926

(22) Filed: May 28, 2009

Related U.S. Application Data
(60) Provisional application No. 61/157,354, filed on Mar. 4, 2009.
(60) Provisional application No. 61/189,154, filed on Aug. 14, 2008.
(60) Provisional application No. 61/137,947, filed on Aug. 4, 2008.
(60) Provisional application No. 61/134,881, filed on Jul. 14, 2008.
(60) Provisional application No. 61/134,653, filed on Jul. 10, 2008.

(51) Int. Cl. C07D 471/04

(52) U.S. Cl. 544/343

(57) ABSTRACT

Processes for purifying Varenicline base or the L-tartrate salt thereof and for preparing Varenicline L-tartrate crystalline forms A and B are provided.

### RELATED APPLICATIONS

This application claims benefit of U.S. Provisional Patent Applications Nos. 61/157,354, filed Mar. 4, 2009, 61/189,154, filed Aug. 14, 2008, 61/137,947, filed Aug. 4, 2008, 61/134,881, filed Jul. 14, 2008, and 61/134,653, filed Jul. 10, 2008, the contents of which are incorporated herein in their entirety by reference.

### FIELD OF INVENTION

The present invention is directed to processes for purifying Varenicline base and Varenicline L-tartrate salt and preparing crystalline forms A and B of Varenicline L-tartrate salt (VRN L-tartrate).

### BACKGROUND OF THE INVENTION

Varenicline tartrate salt, 7,8,9,10-tetrahydro-6,10-methano-6H-pyrazino[2,3-h][3]benzazepine, (2R,3R)-2,3-dihydroxybutanedioate (1:1) has the following formula:

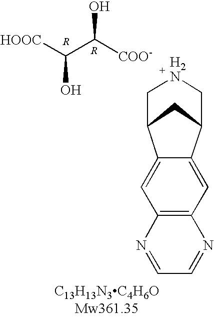

Varenicline tartrate is marketed by Pfizer under the trade name of CHANTIX™ as a partial agonist selective for certain subtypes of nicotinic receptors and indicated for smoking cessation.

Varenicline base and various salts thereof are described in the U.S. Pat. No. 6,410,550, EP 1044189, EP 1659114, and EP1866308.

Varenicline L-tartrate and its crystalline forms A, characterized by XRPD peaks at 6.1, 12.2, 13.0, 14.7, 16.8, 19.4, 21.9, 24.6; B, characterized by XRPD peaks at 5.9, 12.8, 14.4, 15.3, 16.9, 17.2, 21.8, 23.8, 25.1; and C, characterized by XRPD peaks at 5.9, 11.8, 16.5, 21.2, 23.1, 23.8, 26.5 are described in the U.S. Pat. Nos. 6,890,927 and 7,265,119.

WO 2008/060487 describes crystalline forms of Varenicline base and intermediates thereof.

U.S. Pat. No. 6,897,310 describes a nitro intermediate and U.S. Pat. No. 6,951,938 describes an amino intermediate of Varenicline.

U.S. Patent Application Publication No. 2007/0224690 describes Varenicline having 0 to 500 ppm of several impurities.

Polymorphism, the occurrence of different crystal forms, is a property of some molecules and molecular complexes. A single molecule, like Varenicline (VRN) L-tartrate, may give rise to a variety of crystalline forms having distinct crystal structures and physical properties. The difference in the physical properties of different crystalline forms results from the orientation and intermolecular interactions of adjacent molecules or complexes in the bulk solid. Accordingly, polymorphs are distinct solids sharing the same molecular formula yet having distinct advantageous physical properties compared to other crystalline forms of the same compound or complex.

The present invention relates to the solid state physical properties of Varenicline L-tartrate. These properties can be influenced by controlling the conditions under which Varenicline L-tartrate is obtained in solid form. Solid state physical properties include, for example, the flow-ability of the milled solid. Flow-ability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch, or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

There is a need in the art for new processes for preparing polymorphic forms of Varenicline L-tartrate and for additional processes for purifying Varenicline L-tartrate.

### SUMMARY OF THE INVENTION

In one embodiment, the present invention provides a process for purifying Varenicline base or L-tartrate salt thereof, comprising filtering an aqueous solution, ethanol-water solution, methanolic solution, or mixtures thereof of Varenicline base or L-tartrate salt thereof in the presence of activated carbon, wherein filtering is performed using a filter aid.

Optionally, when the Varenicline used in the process of the present invention is Varenicline L-tartrate salt, spray drying is further performed on the obtained salt.

In a preferred embodiment, the Varenicline L-tartrate subjected to filtration described in the present invention is prepared by a process comprising: a) combining a solution of Varenicline base in methanol with L-tartaric acid to obtain a compound of Varenicline L-tartrate, and b) drying the obtained compound to obtain Varenicline L-tartrate. Preferably, the Varenicline L-tartrate obtained is in an amorphous form.

Optionally, the filtrated Varenicline base obtained in the process of present invention is combined with methanolic L-tartaric acid to obtain Varenicline L-tartrate.

In one embodiment, the present invention provides a process for preparing Varenicline L-tartrate crystalline form A, comprising dissolving Varenicline L-tartrate in water, and precipitating Varenicline L-tartrate form A by adding the aqueous solution of Varenicline L-tartrate to an anti-solvent.

In another embodiment, the present invention provides a process for preparing Varenicline L-tartrate crystalline form B, comprising dissolving Varenicline L-tartrate in water, and precipitating Varenicline L-tartrate form B by adding an anti-solvent to the aqueous solution of Varenicline L-tartrate, wherein the water used is not more than 1.5 percent of the total volume.

In yet another embodiment, the present application provides a process for preparing Varenicline L-tartrate crystalline form B, comprising combining Varenicline base, L-tartaric acid, and an ethanol-water solution to precipitate Varenicline L-tartrate crystalline form B.

### DETAILED DESCRIPTION OF THE INVENTION

The present invention relates to purification processes of Varenicline base and Varenicline L-tartrate salt using filtration and activated carbon.

Filtration can be performed using filter aids. The filter aids and other reagents used in the process of the present application are commercially available and suitable for industrial scale production.

As used herein, the term “filter aids” refers to inert porous solids, such as, e.g., those made primarily of silica or wood cellulose, which are used to aid filtration. Examples of filter aids that may be used in the present invention include tonsil beds, hyflow beds, celite functional filters, and macro-cell functional filters. A tonsil bed is typically an acid-activated calcium Bentonite (an absorbent aluminum phyllosilicate, generally impure clay consisting mostly of montmorillonite). A sinter bed is typically a finely perforated glass filter in which a filer aid, such as tonsil bed, may be adapted.

The present invention also relates to processes for preparing crystalline forms A and B of Varenicline L-tartrate salt.

In one embodiment, the present invention provides a process for purifying Varenicline base or L-tartrate salt thereof, comprising filtering an aqueous solution, ethanol-water solution, methanolic solution or mixtures thereof of Varenicline base or L-tartrate salt thereof in the presence of activated carbon, wherein filtering is performed using a filter aid

The filter aid used in the purification process described above is preferably selected from a group consisting of tonsil beds, hyflow beds, celite functional filters, and macro-cel functional filters. More preferably, the filter aid is a tonsil bed.

Preferably, the filter aid is used with vacuum filtration under reduced pressure. More preferably, the reduced pressure is between about 10 mbar to about 100 mbar. Most preferably it is about 30 mbar.

The Varenicline L-tartrate salt obtained according to the above process is obtained with purity greater than about 99.6 percent by area HPLC. Preferably, it is obtained with purity greater than about 99.9 percent. Most preferably it is obtained with a purity of about 100 percent.

When the Varenicline used in the above process is Varenicline L-tartrate salt, spray drying is preferably further performed on the obtained salt.

The term “spray drying” broadly refers to processes involving breaking up liquid mixtures into small droplets (atomization), and rapidly removing solvent from the mixture. In a typical spray drying apparatus, there is a strong driving force for evaporation of solvent from the droplets, which may be provided by providing a drying gas. Spray drying processes and equipment are described in Perry's Chemical Engineer's Handbook, pgs. 20-54 to 20-57 (Sixth Edition 1984), which is incorporated herein by reference.

By way of non-limiting example only, the typical spray drying apparatus comprises a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream of the drying chamber. Examples of such apparatuses include Niro Models PSD-1, PSD-2 and PSD-4 (Niro A/S, Soeborg, Denmark), and BUCHI Model B-290 mini spray dryer.

As used herein, an “inlet temperature” is the temperature at which the drying gas enters the spray dryer; an “outlet temperature” is the temperature at which the gas exits the spray dryer.

Inlet or outlet temperatures may be varied, if necessary, depending on the equipment, gas, or other experimental parameters. For example, it is known that the outlet temperature may depend on parameters such as aspirator rate, air humidity, inlet temperature, spray air flow, feed rate, concentration, or a combination thereof.

When spray drying is performed in the process of the present invention, the inlet temperature is typically between about 180° C. to about 230° C., and, preferably, about 190° C. to about 220° C. More preferably, the inlet temperature is about 213° C. to about 220° C. The outlet temperature is typically about 105° C. to about 130° C., and, preferably, about 113° C. to 120° C. More preferably the outlet temperature is about 117° C. to about 119° C.

Typically, the product collection means includes a cyclone connected to the drying apparatus. In the cyclone, the particles produced during spray drying are separated from the drying gas and evaporated solvent, allowing the particles to be collected. A filter may also be used to separate and collect the particles produced by spray drying. Spray-drying may be performed in a conventional manner in the processes of the present invention (see, e.g., Remington: The Science and Practice of Pharmacy, 19th ed., vol. II, pg. 1627, herein incorporated by reference). The drying gas used in the invention may be any suitable gas, although inert gases such as nitrogen, nitrogen-enriched air, and argon are preferred. Nitrogen gas or air is a particularly preferred drying gas for use in the process of the invention. The amorphous Varenicline L-tartrate product produced by spray-drying may be recovered by techniques commonly used in the art, such as by using a cyclone or a filter.

Preferably, methanolic L-tartaric acid is added when an aqueous solution of Varenicline base is filtered.

In a preferred embodiment, a powdery compound of Varenicline L-tartrate is first obtained by addition of L-tartaric acid to a solution of Varenicline base in methanol, the wet material is dried, and dissolved in water in the presence of activated carbon (CXV). The resulting mixture is then filtered using a sinter and tonsil bed under reduced pressure, and the solvent is removed by spray-drying to give purified Varenicline L-tartrate in an amorphous form. Preferably, the Varenicline L-tartrate obtained according to the above preferred process is obtained with a purity of about 100 percent by area HPLC.

In one specific embodiment, the aqueous solution of Varenicline base used in the above process also contains methanol. Typically, L-methanolic tartaric acid is further added to obtain pure Varenicline L-tartrate salt.

In another specific embodiment, activated carbon (CXV) is added to Varenicline base in methanol, the obtained mixture is filtered under reduced pressure using a sinter and tonsil bed, a methanolic solution of L-tartaric acid is added to the filtered Varenicline base solution, and a precipitate of Varenicline L-tartrate is obtained.

In one embodiment, the present invention provides a process for preparing Varenicline L-tartrate crystalline form A, comprising dissolving Varenicline L-tartrate in water, and precipitating Varenicline L-tartrate form A by adding the aqueous solution of Varenicline L-tartrate to an anti-solvent.

Preferably, when the aqueous solution of Varenicline L-tartrate is added to the anti-solvent, it is added drop-wise.

Preferably, the aqueous solution of Varenicline L-tartrate is added at a temperature of about 50° C. to about 80° C. More preferably, it is added at a temperature of about 70° C.

The volume ratio between the anti-solvent and the water used in the process described above is between about 1:15 to about 1:35 (v/v) of water:anti-solvent. More preferably, the ratio is between about 1:20 to about 1:35. Most preferably, it is about 1:33 (v/v).

In another embodiment, the present invention provides a process for preparing Varenicline L-tartrate crystalline form B, comprising dissolving Varenicline L-tartrate in water, and precipitating Varenicline L-tartrate form B by adding an anti-solvent to the aqueous solution of Varenicline L-tartrate, wherein the water used is not more than 1.5 percent of the total volume.

Preferably, the water is used at 1.4 percent, more preferably at 1.3 percent of the total volume.

The volume ratio between the anti-solvent and the water used in the process described above is between about 1:5 to about 1:10 (v/v) of water:anti-solvent. More preferably, the ratio is between about 1:8 to about 1:10. Most preferably, it is about 1:9 (v/v).

The anti-solvent used in any of the processes described above is selected from a group consisting of C₁-C₄ alcohols, tetrahydrofuran (THF), and acetonitrile. Preferably, the anti-solvent is selected from a group consisting of ethanol or isopropanol, THF and acetonitrile. Most preferably, the anti-solvent is ethanol.

In yet another embodiment, the present application provides a process for preparing Varenicline L-tartrate crystalline form B, comprising combining Varenicline base, L-tartaric acid, and ethanol-water solution to precipitate Varenicline L-tartrate crystalline form B.

The ethanol-water solution in the process described above is at a volume ratio of about 90 percent:10 percent (9:1) to about 98 percent:2 percent (49:1) of ethanol:water (v/v). Preferably, the ratio is about 92 percent:8 percent (11.5:1) (v/v) to about 96 percent: 4 percent (24:1) (v/v), and more preferably, it is about 95 percent:5 percent (19:1) (v/v)

The reaction mixture described above contains Varenicline base and ethanol-water at a ratio of about 10:1 to about 5:1 of ethanol-water:Varenicline base (v/w). Preferably, the ratio is about 9:1 to about 7:1 (v/w), and more preferably the ratio is about 7.5:1 (v/w).

Optionally, Varenicline base is reacted with activated carbon (CVX) prior to its addition to the reaction mixture.

Varenicline L-tartrate form B used in any of the above processes can be obtained according to any method known in the art, for example in U.S. Pat. Nos. 6,890,927 and 7,265,119, incorporated herein by reference, wherein L-tartaric acid in methanol was combined with Varenicline base in methanol, or according to examples 3 and 10 of the present application.

Varenicline base used in any of the above processes may be obtained according to any method known in the art, for example in U.S. Pat. No. 6,410,550 incorporated herein by reference, wherein 1-(5,8,14-Triazatetracyclo[10.3.1.0^{2,11}.0^{4,9}]hexadeca-2(11),3,5,9-pentaene)-2,2,2-trifluoro-ethanone in methanol is reacted with a base, e.g., alkali metal, alkaline earth metal carbonates or hydroxides, and then heated, or according to the first part of example 3 of the present application.

Having described the invention with reference to certain preferred embodiments, other embodiments will become apparent to one skilled in the art from consideration of the specification. The disclosures of the references referred to in this patent application are incorporated herein by reference. The invention is further defined by reference to the following examples describing in detail the process and compositions of the invention. It will be apparent to those skilled in the art that many modifications, both to materials and methods, may be practiced without departing from the scope of the invention.

### EXAMPLES

### Experimental Methodology

HPLC

| Column & packing | Chromatopak C18 150, 4.6 mm, 5μ P.N 1546511 | | |
|---|---|---|---|
| Eluent | A - 75% - 0.02 M NH₄H₂PO₄ adjusted to pH = 6.0 | | |
| | with NH₄OH | | |
| | 25% - MeOH | | |
| | B - 20% - 0.02 M NH₄H₂PO₄ adjusted to pH = 6.0 | | |
| | with NH₄OH | | |
| | 80% - MeOH | | |
| | | A | B |
| Gradient | 0 | 100 | 0 |
| | 5 min | 100 | 0 |
| | 30 min | 0 | 100 |

| | |
|---|---|
| Stop time: | 30 min |
| Flow: | 1.0 ml/min |
| Detector: | 210 nm. |
| Injection volume: | 10 μl. |
| Diluent | Eluent A |
| Column temperature | 25° C. |

### Sample Solution Preparation:

Weigh accurately about 20 mg of Varenicline Tartrate sample into a 20 ml volumetric flask, dissolve, and dilute to volume with diluent. Dilute 5 ml into a 10 ml volumetric flask with diluent.

### Method:

Inject sample solutions continuing the chromatogram up to the end of gradient. Determine the area of each impurity using suitable integrator.

### Calculations:

Any impurity in a sample is calculated as follows:

$$\% Impurity in sample = \frac{area impurity in sample}{\sum Areas of all peaks} \times 100$$

### Water Content

Water content was determined by Karl Fisher (KF) analysis using Mettler Toledo DL 38 Karl Fisher Titrator.

### Example 1

### Purification of Varenicline L-Tartrate

### Method 1:

3.15 g of L-Tartaric acid was dissolved in 34 ml of water. 4 g of Varenicline base was added at 25°±5° C. for 5 minutes to get a clear solution. To the clear solution, 0.8 g activated carbon (CXV) was added, and stirred at 25°±5° C. for 30 minutes. The mixture was vacuum filtered under reduced pressure with a Sinter and tonsil. Then the solution was spray dried to obtain amorphous Varenicline L-Tartrate. The nitrogen gas was at an inlet temperature of 220° to 190° C. The evaporated solvent and nitrogen left the spray dryer at a temperature of 113° to 120° C.

The impurity profile obtained by HPLC analysis of Method 1 is provided in table 1

TABLE 1

| | Impurity profile by HPLC (% area) RT/RRT | | | | | | | | | |
|---|---|---|---|---|---|---|---|---|---|---|
| | | 3.07/ | 3.72/ | | | 5.90/ | 10.27/ | | | 21.8/ |
| | 0.97 | 3.86 | 5.39 | 4.53 | 5.32 | 7.71 | 9.77 | 10.86 | 16.17 | 23.8 |
| Description | 0.18 | 0.82 | 1.00 | 1.22 | 1.43 | 1.59 | 2.76 | 2.92 | 4.35 | 5.86 |
| VRN Base | ND | 0.11 | 98.29 | 0.02 | 0.08 | 0.57 | 0.05 | 0.38 | 0.22 | 0.29 |
| VRN | ND | ND | 99.95 | ND | ND | ND | ND | ND | ND | 0.05 |
| Tartrate | | | | | | | | | | |

### Method 2:

A. 20 g of Varenicline base were dissolved in 150 ml of methanol, and stirred for 20 minutes. To the obtained solution, 15.7 g of L-tartaric acid dissolved in 150 ml methanol was added at 25°±5° C. for 30 to 40 minutes to precipitate Varenicline L-Tartrate. The mixture was stirred at 25°±5° C. for 2 to 20 hours, filtered, and washed with 70 ml methanol to get a powdery compound. The wet material was dried under vacuum at T≦50° C.
B. 5 g of Varenicline L-Tartrate were dissolved in 40 ml water at about 25°+5° C. To the clear solution, 1 g activated carbon (CXV) was added, and stirred at 25°±5° C. for 30 minutes. The mixture was vacuum filtered under reduced pressure with a Sinter and tonsil. Then, the solution was spray dried to obtain amorphous Varenicline L-Tartrate. The nitrogen gas was at an inlet temperature of 220° to 213° C. The evaporated solvent and nitrogen left the spray dryer at a temperature of 117° to 119° C.
The impurity profile obtained by HPLC analysis of Method 2 is provided in table 2

TABLE 2

Impurity profile by HPLC
(% area) RT/RRT

| | 4.44 | 4.7 | 6.0 | 22.3 |
|---|---|---|---|---|
| | 1.00 | 1.4 | 1.44 | 5.36 |
| VRN Tartrate | 99.65 | ND | 0.12 | 0.2 |
| After spray-dryer | 100 | ND | ND | ND |

### Example 2

### Purification of Varenicline L-Tartrate

### Method 3:

25 g of Varenicline base were dissolved in 150 ml methanol. To the clear solution activated carbon (CXV) was added, and the obtained mixture was stirred at 25°±5° C. for 30 minutes. The mixture was vacuum filtered under reduced pressure with a Sinter and tonsil bed. The Varenicline base solution was added to a methanolic solution of L-Tartaric acid (19.7 g) (dissolved in 7.5 volume of methanol relative to Varenicline base) at 25°±5° C. for 10 to 40 minutes to precipitate Varenicline L-Tartrate. The mixture was stirred at 25°±5° C. for 2 to 20 hours, filtered, and washed with 70 ml of methanol to get a powdery creamy solid. The wet material was dried under vacuum at Tj=50° C.

### Method 4:

25 g of Varenicline base was dissolved in 150 ml methanol and 6 ml water. To the clear solution activated carbon (CXV) was added, and the obtained mixture was stirred at 25°±5° C. for 30 minutes. The mixture was vacuum filtered under reduced pressure with a Sinter and tonsil bed. The Varenicline base solution was added to a methanolic solution of L-Tartaric acid (19.7 g) (dissolved in 7.5 volume of methanol relative to Varenicline base) at 25°±5° C. for 10 to 40 minutes to precipitate Varenicline L-Tartrate. The mixture was stirred at 25°±5° C. for 2 to 20 hours, filtered, and washed with 70 ml of methanol to get a powdery white to off-white solid. The wet material was dried under vacuum at Tj=50° C.

The impurity profile obtained by HPLC analysis of Methods 3 and 4 is provided in Table 3.

TABLE 3

| | Impurity profile by HPLC (% area) RRT | | | | | | | | | | |
|---|---|---|---|---|---|---|---|---|---|---|---|
| Description | Yield % | 0.82q | 1.00 | 1.22 | 1.43 | 1.59 | 2.76 | 2.92 | 4.35 | 5.86 | ASSAY % |
| VRN Base | NA | 0.11 | 98.29 | 0.02 | 0.08 | 0.57 | 0.05 | 0.38 | 0.22 | 0.29 | 90.3% |
| Method 3- | 86% | 0.03 | 99.62 | ND | ND | 0.05 | 0.08 | ND | ND | 0.23 | 98.36 |
| Crystallization | | | | | | | | | | | |
| from Methanol | | | | | | | | | | | |
| Method 4- | 60% | ND | 99.91 | ND | ND | 0.03 | 0.02 | ND | ND | 0.03 | 101.9% |
| Crystallization | | | | | | | | | | | |
| from Methanol | | | | | | | | | | | |
| and water (4% | | | | | | | | | | | |
| v/v vs. | | | | | | | | | | | |
| Methanol) | | | | | | | | | | | |

### Example 3

### Reference Example: Example 4 of EP 1866308: Preparation of Varenicline L-Tartrate Form B

A clean, dry 4 neck round bottom flask equipped with mechanical stirrer and thermo pocket was charged with toluene (119.0 ml), 1-(5,8,14-triazatetracyclo[10.3.1.0^{2,11}.0^{4,9}]hexadeca-2(11),3,5,9-pentane)-2,2,2-trifluoro-ethanone (17.0 gm) at 25-30° C., which was treated with 2N aqueous solution of sodium hydroxide (86.03 ml) with stirring. The mixture was warmed to 30° to 35° C. for 2 hours, and progress of the reaction was monitored by HPLC/TLC (MDC:MeOH 9:1). After completion of the reaction, toluene (170 ml) was added to the reaction mixture, and stirred for 20 minutes. The layers were separated, and the aqueous layer was extracted with toluene (2×85 ml). Combined organic layer was distilled to remain in residue up to 5 volumes. To the above solution, methanol (255 ml) was charged and azeotropically distilled under vacuum up to 5 volumes (85 ml). Methanol (170 ml) was charged, and again azeotropically distilled under vacuum up to 5 volumes (85 ml). Methanol (305 ml) was added in the remaining methanolic solution, which was further treated with activated carbon (1.7 gm) for 1 hour at 25° to 30° C. Filtered the solution through celite bed and transferred to an addition funnel.

In a separate clean and dry 4-neck round bottom flask, L-(+)-Tartaric acid (9.14 gm) was dissolved in methanol (221 ml) at 25° to 30° C. To this above methanolic solution, Varenicline base was added drop wise in 20 to 30 minutes through an addition funnel. The resulting precipitate was stirred for 1 hour, filtered, and washed with methanol (34 ml) to afford the product i.e. 5,8,14Triazatetracyclo[10.3.1.0^{2,11}.0^{4,9}]hexadeca-2(11),3,5,7,9-pentaene Tartrate salt (Varenicline L-Tartrate). (Yield 15.7 gm, HPLC Purity NLT-99.5 percent).

### Preparation of Varenicline L-Tartarate Form A

### Example 4

Varenicline L-tartrate Form B (0.15 g, obtained in example 3) was dissolved in water (3 vol. 0.45 ml) at 70° C. The solution was added drop-wise into Isopropanol (100 vol, 15 ml), and precipitation occurred. The slurry was stirred 48 hours, filtered and dried in 55° C. vacuum oven. A PXRD analysis confirmed the product was Varenicline L-tartrate Form A.

### Example 5

Varenicline L-tartrate Form B (0.15 g, obtained in example 3) was dissolved in water (3 vol, 0.45 ml) at 70° C. the solution was added drop-wise into Ethanol (100 vol, 15 ml) and precipitation occurred. The slurry was stirred 48 hours, filtered and dried in 55° C. vacuum oven. A PXRD analysis confirmed the product was Varenicline L-tartrate Form A.

### Preparation of Varenicline L-Tartarate Form B

### Example 6

Varenicline L-tartrate Form B (0.15 g, obtained in example 3) was dissolved in water (10 vol, 1.5 ml at 70° C. Acetonitrile (66 vol, 10 ml) was added, and precipitation occurred. The slurry was cooled to room temperature, stirred 16 hours, filtered, and dried in 55° C. vacuum oven. A PXRD analysis confirmed the product was Varenicline L-tartrate Form B.

### Example 7

Varenicline L-tartrate Form B (0.15 g, obtained in example 3) was dissolved in water (10 vol, 1.5 ml) at 70° C. Ethanol (93 vol, 14 ml) was added, and precipitation occurred. The slurry was cooled to room temperature, stirred 16 hours, filtered, and dried in 55° C. vacuum oven. A PXRD analysis confirmed the product was Varenicline L-tartrate Form B.

### Example 8

Varenicline L-tartrate Form B (0.15 g, obtained in example 3) was dissolved in water (10 vol, 1.5 ml) at 70° C. Isopropanol (93 vol, 14 ml) was added, and precipitation occurred. The slurry was cooled to room temperature, stirred 16 hours, filtered, and dried in 55° C. vacuum oven. A PXRD analysis confirmed the product was Varenicline L-tartrate Form B.

### Example 9

Varenicline L-tartrate Form B (0.15 g, obtained in example 3) was dissolved in water (10 vol, 1.5 ml) at 70° C. Tetrahydrofuran (80 vol, 12 ml) was added, and precipitation occurred. The slurry was cooled to room temperature, stirred 16 hours, filtered, and dried in 55° C. vacuum oven. A PXRD analysis confirmed the product was Varenicline L-tartrate Form B.

### Example 10

120 g of Varenicline base were dissolved in 900 ml Ethanol 95 percent (5 percent water). To the clear solution activated carbon (CXV) was added, and the obtained mixture was stirred at 25±5° C. for 30 minutes. The mixture was filtered under reduced pressure with Sinter, and washed with Ethanol 95 percent (5 percent water).

The Varenicline base solution was added to a solution of L-Tartaric acid (94.44 g) in Ethanol 95 percent (5 percent water) 900 ml (dissolved in 7.5 volume of Ethanol 95 percent (5 percent water) relate to Varenicline base) at 25±5° C. for 10 to 40 minutes to get a precipitation of Varenicline L-Tartarate. The mixture was stirred at 25±5° C. for 2 to 20 hours, filtered, and washed with 240 ml of Ethanol 95 percent (5 percent water) to get a powdery white to off-white solid. The wet material was dried under vacuum at Tj=50° C., to obtain Form B.

| | |
|---|---|
| Impurity profile by | |
| HPLC (% area) | L- |
| RRT | Tartaric |

| 7.7 | 17.4 | | | Acid | |
|---|---|---|---|---|---|
| 1.0 | 2.27 | Ash | KF | (%) | ASSAY % |
| 99.95 | 0.05 | 0.02 | 0.36 | 41.6 | 101.9 |

We claim:

1. A process for purifying Varenicline base or L-tartrate salt thereof, comprising filtering an aqueous solution, ethanol-water solution, methanolic solution, or mixtures thereof of Varenicline base or L tartrate-salt thereof in the presence of activated carbon, wherein the filtering comprises the use of a filter aid.

2. The process of claim 1, wherein the filter aid is selected from a group consisting of a tonsil bed, hyflow bed, celite functional filter, and macro-cel functional filter.

3. The process of claim 2, wherein the filter aid is a tonsil bed.

4. The process of claim 1, wherein the filtering with the filter aid is vacuum filtration at a pressure of about 10 mbar to about 100 mbar.

5. The process of claim 4, wherein the pressure is about 30 mbar.

6. The process of claim 1, wherein the obtained Varenicline L-tartrate salt has a purity greater than about 99.6 percent by area HPLC.

7. The process of any of claim 6, wherein the obtained Varenicline L-tartrate salt has a purity greater than about 99.9 percent by area HPLC.

8. The process of claim 7, wherein the obtained Varenicline L-tartrate salt has a purity of about 100 percent by area HPLC.

9. The process of claim 1, wherein the resulting filtered solution of Varenicline L-tartrate salt is spray dried.

10. The process of claim 9, wherein the Varenicline L-tartrate salt is spray dried at an inlet temperature of about 180° C. to about 230° C. and an outlet temperature of about 105° C. to about 130° C.

11. The process of claim 9, wherein the Varenicline L-tartrate salt is spray dried at an inlet temperature of about 190° C. to about 220° C. and an outlet temperature of about 113° C. to about 120° C.

12. The process of claim 9, wherein Varenicline L-tartrate salt is spray dried at an inlet temperature of about 213° C. to about 220° C. and an outlet temperature of about 117° C. to about 119° C.

13. The process of claim 9, further comprising preparing the Varenicline L-tartrate subjected to filtration in a process comprising: a) combining a solution of Varenicline base in methanol with L-tartaric acid to obtain Varenicline L-tartrate and b) drying the obtained Varenicline L-tartrate

14. The process of claim 13, wherein the Varenicline L-tartrate obtained is amorphous.

15. The process of claim 13, wherein the obtained Varenicline L-tartrate has a purity of about 100 percent by area HPLC.

16. The process of claim 1, further comprising combining the filtered Varenicline base with methanolic L-tartaric acid to obtain Varenicline L-tartrate.

17. A process for preparing Varenicline L-tartrate crystalline form A, comprising dissolving Varenicline L-tartrate in water, adding the resulting aqueous solution of Varenicline L-tartrate to an anti-solvent, and precipitating Varenicline L-tartrate form A.

18. The process of claim 17, wherein the aqueous solution of Varenicline L-tartrate is added drop-wise into the anti-solvent.

19. The process of claim 17, wherein the aqueous solution of Varenicline L-tartrate is added at a temperature of about 50° C. to about 80° C.

20. The process of claim 19, wherein the anti-solvent is selected from a group consisting of C₁-C₄ alcohols, tetrahydrofuran, and acetonitrile.

21. The process of claim 17, wherein the anti-solvent and water have a volume ratio between about 1:15 and about 1:40 (v/v) of water:anti-solvent.

22. The process of claim 17, wherein the anti-solvent and water have a volume ratio between about 1:15 and about 1:35 (v/v) of water:anti-solvent.

23. The process of claim 17, wherein the volume ratio between the anti-solvent and the water is about 1:33 (v/v) of water:anti-solvent.

24. A process for preparing Varenicline L-tartrate crystalline form B, comprising dissolving Varenicline L-tartrate in water, adding an anti-solvent to the resulting aqueous solution of Varenicline L-tartrate, and precipitating Varenicline L-tartrate form B, wherein the water is used in an amount not more than 1.5 percent of the total volume.

25. The process of claim 24, wherein the water is used in an amount not more than 1.3 percent of the total volume.

26. The process of claim 24, wherein the anti-solvent and the water have a volume ratio between about 1:5 to about 1:10 (v/v) of water:anti-solvent.

27. The process of claim 24, wherein the anti-solvent and the water have a volume ratio between about 1:8 to about 1:10 (v/v) of water:anti-solvent.

28. The process of claim 24, wherein the anti-solvent and the water have a volume ratio between about 1:9 (v/v) of water:anti-solvent.

29. The process of claim 17, wherein the anti-solvent is selected from a group consisting of C₁-C₄ alcohols, tetrahydrofuran, and acetonitrile.

30. The process of claim 17, wherein the anti-solvent is selected from a group consisting of ethanol, isopropanol, tetrahydrofuran, and acetonitrile.

31. The process of claim 17, wherein, the anti-solvent is ethanol.

32. A process for preparing Varenicline L-tartrate crystalline form B, comprising combining Varenicline base, L-tartaric acid, and an ethanol-water solution to precipitate Varenicline L-tartrate crystalline form B.

33. The process of claim 32, wherein the ethanol-water solution has a volume ratio of about 90 percent:10 percent (9:1) to about 98 percent:2 percent (49:1) of ethanol:water (v/v).

34. The process of claim 33, wherein the ethanol-water mixture has a volume ratio of about 95 percent:5 percent (19:1) (v/v).

35. The process of claim 32, wherein the Varenicline base and ethanol-water are combined in a ratio of about 10:1 to about 5:1 of ethanol-water:Varenicline base (v/w).

36. The process of claim 35, wherein the Varenicline base and ethanol-water are combined in a ratio of about 7.5:1 (v/w).
